(19) United States
(12) Patent Application Publication
(10) Pub. No.: US 2014/0060540 A1
(43) Pub. Date: Mar. 6, 2014

(54) METHOD AND SYSTEM FOR AUTOMATED SPONTANEOUS BREATHING TRIAL

(71) Applicants: Gary S. Milne, Louisville, CO (US); Robert T. Boyer, Longmont, CO (US)

(72) Inventors: Gary S. Milne, Louisville, CO (US); Robert T. Boyer, Longmont, CO (US)

(73) Assignee: NELLCOR PURITAN BENNETT LLC, Boulder, CO (US)

(21) Appl. No.: 13/604,901

(22) Filed: Sep. 6, 2012

(51) Int. Cl. A61M 16/00

(52) U.S. Cl. 128/204.23

(57) ABSTRACT

The disclosure relates generally to a method and a system for acquiring patient parameters of a plurality of patient parameter relationship expressions and performing an automated spontaneous breathing trial. In the event of a successful trial, constructive notice of the success is provided.

### TECHNICAL FIELD

The disclosure relates generally to a method and a system for monitoring a patient's unassisted breathing readiness. More particularly, the disclosure relates to a method and a system for automatically performing a spontaneous breathing trial.

### BACKGROUND

Ventilators provide pressurized respiratory gases to patients to assist their breathing. Respiratory gases may include fresh air, scrubbed air, and anesthetics, for example. As the patient conditions change, the patient gradually transitions from assisted to unassisted breathing. A patient's readiness for withdrawal from mechanical ventilation is typically indicated by the successful completion of a spontaneous breathing trial (SBT). The discontinuation of assisted ventilation process may require as much as 42% of the time that a patient spends on a mechanical ventilator. Explicitly performed SBTs, which are performed by a clinician, limit the frequency at which a patient may be tested. Unnecessary delays in the discontinuation process may increase the complication rate from mechanical ventilation, e.g. pneumonia and airway trauma.

Even further, it may be beneficial to determine when a patient is not ready to breathe unassisted. Aggressiveness in the discontinuation process can cause additional problems, such as difficulty in reestablishing artificial airways and compromising gas exchange. More favorable patient outcomes and lower costs may be possible by earlier transition to unassisted breathing.

### SUMMARY

A method and a system to automatically perform a spontaneous breathing trial are provided herein. In one embodiment according to the disclosure, a method implemented with a computing system comprises acquiring patient parameters of patient parameter relationship expressions stored in a computer readable storage medium; evaluating the patient parameter relationship expressions; determining if a success criteria is satisfied, the success criteria based at least in part on satisfaction of at least some of the plurality of patient parameter relationship expressions for a given time; and determining that the spontaneous breathing trial is successful if the success criteria is satisfied.

The above-mentioned and other disclosed features which characterize the embodiments of the system and method described herein advantageously enable standardization of the process of, and protocols for, evaluating a patient's unassisted breathing readiness. Another advantage is that the system and method will enable performance of spontaneous breathing trials without waiting for daily rounds, thereby reducing the amount of time a patient is ventilated. Even further, as standardized data is acquired over time, the automated spontaneous breathing trial protocols may be improved, reducing even further the amount of time patients are ventilated and/or reducing the complications that arise from premature or delayed discontinuation of ventilation. In addition to the patient care benefits, a further benefit is the reduction of costs associated with ventilating a patient when assisted ventilation is not required.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some or none of the enumerated advantages.

### BRIEF DESCRIPTION OF THE DRAWINGS

The above-mentioned and other disclosed features, and the manner of attaining them, will become more apparent and will be better understood by reference to the following description of disclosed embodiments taken in conjunction with the accompanying drawings, wherein:

FIG. 1 is a block diagram of an embodiment of a system according to the disclosure for performing an automated spontaneous breathing trial;

FIG. 2 is a flowchart of an embodiment of a method according to the disclosure for performing an automated spontaneous breathing trial;

FIG. 3 is a block diagram of an embodiment of a software product according to the disclosure operable for performing an automated spontaneous breathing trial;

FIG. 4 is a block diagram of another embodiment of a system according to the disclosure for performing an automated spontaneous breathing trial; and

FIGS. 5 and 6 are exemplary views of an embodiment of a user interface according to the disclosure.

Corresponding reference characters indicate corresponding parts throughout the several views. Although the drawings represent embodiments of various features and components according to the present disclosure, the drawings are not necessarily to scale and certain features may be exaggerated in order to better illustrate and explain the present invention. The exemplification set out herein illustrates embodiments of the disclosure, and such exemplifications are not to be construed as limiting the scope of the invention in any manner.

### DETAILED DESCRIPTION

For the purposes of promoting an understanding of the principles of the invention, reference will now be made to the embodiments illustrated in the drawings, which are described below. The embodiments disclosed below are not intended to be exhaustive or limit the invention to the precise form disclosed in the following detailed description. Rather, the embodiments are chosen and described so that others skilled in the art may utilize their teachings. It will be understood that no limitation of the scope of the disclosure is thereby intended. The invention includes any alterations and further modifications in the illustrated devices and described methods and further applications of the principles of the invention which would normally occur to one skilled in the art to which the invention relates.

The transitional term “comprising”, which is synonymous with “including,” or “containing,” is inclusive or open-ended and does not exclude additional, unspecified elements or method steps. By contrast, the transitional term “consisting” is a closed term which does not permit addition of unspecified terms.

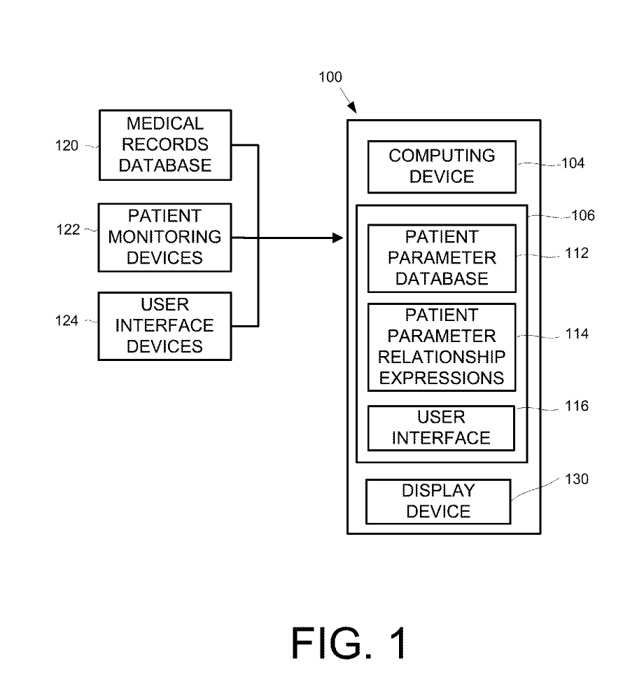

Referring to FIG. 1, a block diagram of an embodiment of a system according to the disclosure for performing an automated spontaneous breathing trial is presented. In the present embodiment, the system is denoted by numeral 100. System 100 may receive patient parameters from a plurality of data sources including a medical records database, patient monitoring devices, and user interfaces. The medical records database may contain patient information such as age and medical condition, and patient parameters based on, for example, laboratory analysis of the patient's fluids. Patient monitoring devices automatically record patient parameters such as heart rate and blood pressure, for example. Patient monitoring devices may be included in, or be part of, a mechanical ventilator. User interfaces enable entry of patient parameters based on, for example, observation of the patient's state or performance of patient maneuvers.

System 100 comprises a computing device 104 and a computer readable storage medium 106 having stored therein a patient parameter database 112, patient parameter relationship expressions 114 and a user interface 116. A medical records database 120, patient monitoring devices 122 and user interface devices 124, are shown coupled to system 100. Computing device 104 may comprise one or more processing device and software programs configured to cause the processing device to acquire patient parameters from the data sources and to store the patient parameters in patient parameter database 112. Each patient parameter relationship expression describes a relationship between at least one patient parameter and a threshold. The relationship comprises an operator and may comprise a single parameter (e.g. A=c, where A is the parameter and “c” is a threshold), more than one parameter (e.g. A+B=c, where A and B are parameters and “c” is a threshold), a function (e.g. f(A)=c, where f(A) is any function of parameter A and “c” a threshold), and any combination of the foregoing. Of course, the operator may represent an inequality as well as an equality relationship. The system may comprise functions which users may include in the expressions. For example, a function may be provided to convert a parameter, and the conversion function may then be used in a patient parameter relationship expression. The expressions may also include ranges, e.g. {A<K+2B≦c}, where A and B are parameters, K is a constant and “c” is a threshold; therefore, the expression may be satisfied by a range of values for A in which A is less than K. Ranges may also be defined in a function comprising Boolean algebra, where the threshold is a logical outcome, e.g. yes/no. Expressions may also include functions representing time durations or compliance requirements. For example, an expression may include a function requiring that a parameter exceed a threshold for a predetermined time during the trial or falls within a range for a predetermined time.

The status of a patient parameter relationship expression is determined by comparing the result of the function to the threshold based on the operator. Thus, the patient parameter relationship expression may be satisfied if the condition specified by the operator is satisfied or unsatisfied if the condition is not satisfied. In the event that the patient parameter is not yet available, the patient parameter relationship expression status may indicate that the evaluation of the expression is incomplete. In the present context, the terms satisfied, unsatisfied and incomplete are only exemplary. Any other suitable terms may be used to denote the status of an expression, protocol or trial. For example, Boolean logic terms such as true/false or yes/no may be used instead of satisfied and unsatisfied.

System 100 may also comprise one or more software programs configured to cause computing device 104 to determine the status of patient parameter relationship expressions 114 based on the patient parameters, to determine if an SBT start criteria is satisfied, to automatically start the SBT if the criteria is satisfied, to determine if an SBT success criteria is satisfied and to indicate a status of the SBT based on the status of the expressions. Exemplary status indicators may comprise icons of different colors or shapes, flashing icons, text messages, audible indications, and any other means for providing constructive notice to the clinician concerning the success or failure of the trial.

In one variation of the present embodiment, a software program may be configured to cause computing device 104 to present status indicators in a predetermined manner with a display device 130. In one example, expression status indicators are presented to indicate which expressions have been satisfied and which have not been satisfied. User interface 116 may also be operable to define expressions and protocols, select and modify the protocols, and associate the protocols with patients. A protocol may be associated with a patient by selecting the patient, selecting the protocol, and then saving or storing the patient configuration file.

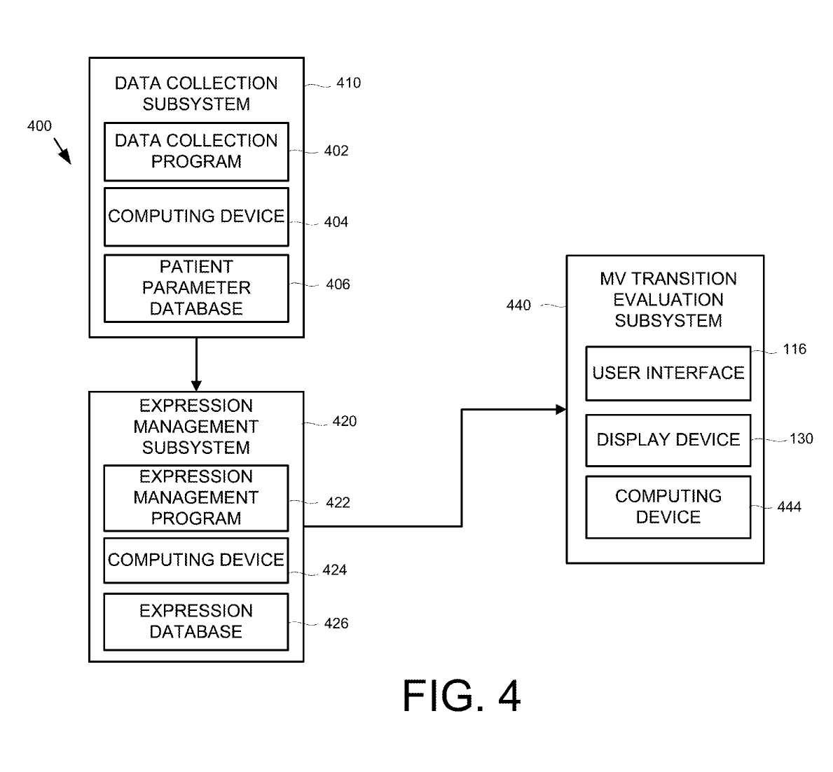

The components of system 100 may be integrated or distributed. A distributed system is described below with reference to FIG. 4.

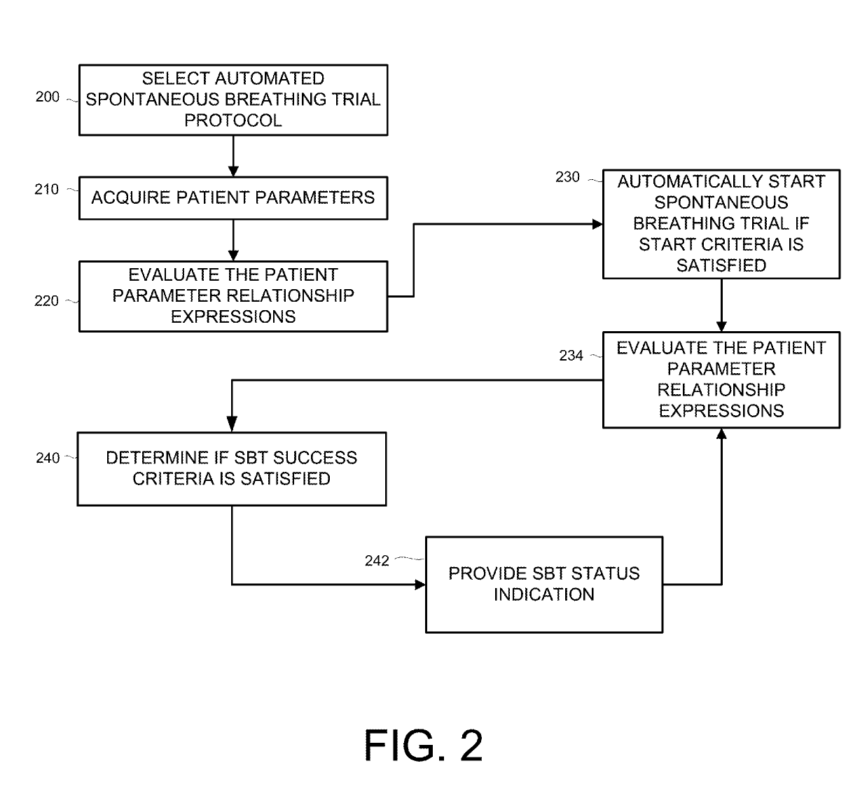

An embodiment of a method according to the disclosure for performing an automated spontaneous breathing trial will now be described with reference to FIG. 2. The method may be performed, at least partially, with system 100. At 200, an automated spontaneous breathing trial protocol is selected. Potentially multiple protocols may be defined (e.g., a well known protocol found in research literature, or a doctor's or hospital's own variant protocol). Each protocol is represented as one or more patient parameter relationship expressions. Also, a protocol may comprise a composite expression comprised of multiple parameters and operators. The protocols may be user configured (e.g., users may add, modify, or delete expressions and protocols as desired—for example to differentiate adult ICU patients having been briefly under anesthesia from a pediatric case from a long-term mechanically ventilated patient). A protocol may include start criteria, success criteria or both success and failure criteria. Success criteria for a protocol may comprise satisfaction of success expressions. Failure criteria for a protocol comprises satisfaction of failure expressions. A protocol may be satisfied when all the success expressions are satisfied and none of the failure expressions are satisfied. In one example, SBT start criteria is satisfied when the success expressions are satisfied and none of the failure expressions are satisfied, at one instance in time, regardless of any duration requirements specified by the expressions.

User interface 116 may be operable to define expressions and protocols, select protocols and modify the protocols. In one variation of the present embodiment, a patient is selected, a predefined protocol is selected, and the predefined protocol is then stored in a patient configuration file or data structure. If a predefined protocol is modified after it is associated with the patient, the modifications may be stored in the patient configuration file. Also, the modifications may be stored in a new protocol. The system may support evaluation of multiple patients by enabling a clinician to switch patients, for example by selecting a different patient from a drop-down list. When the different patient is selected, the different patient's configuration file restores the previously selected protocol associated with the patient.

Once the patient's spontaneous breathing trial protocol is selected, patient parameters are acquired, at 210. Patient parameters may be acquired by the automated spontaneous breathing trial system at periodic time intervals, based on the patient's physiology, or upon occurrence of some predetermined event. Also, patient parameters may be acquired by a data acquisition system, and subsequently the automated spontaneous breathing trial system may, at periodic time intervals, based on the patient's physiology, or upon occurrence of some predetermined event, acquire the patient parameters from the data acquisition system.

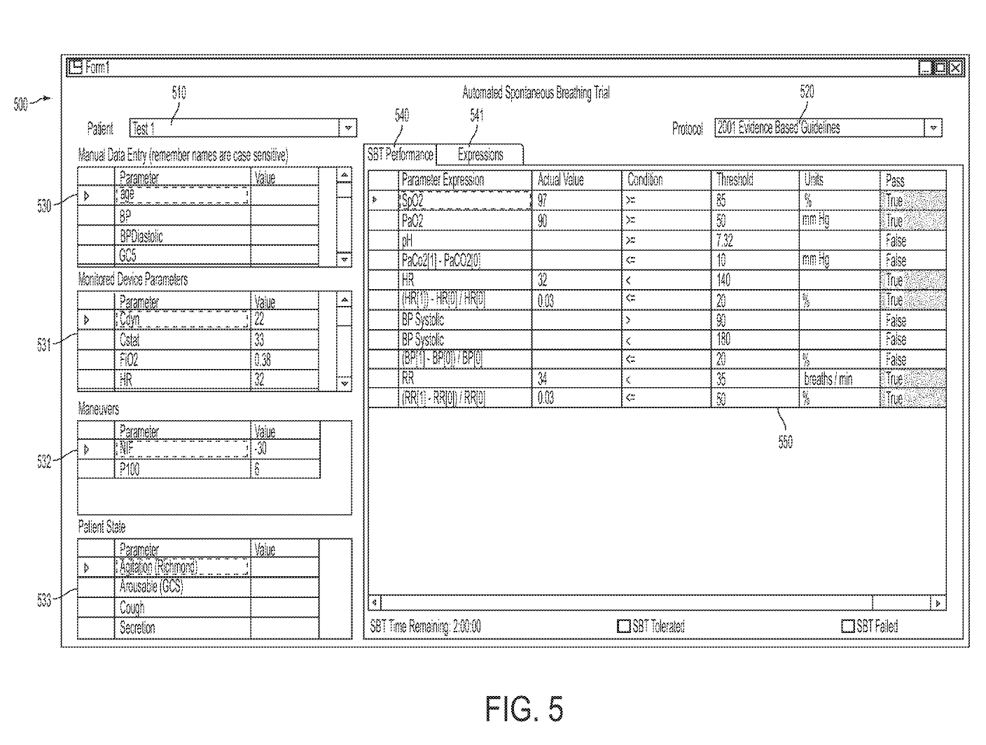

Once the patient parameters have been acquired, the patient parameter relationship expressions are evaluated, at 220 and 234. An exemplary expression based on parameter SpO2 is shown on FIG. 5., where the operator is and the threshold is 85%. Accordingly, the expression is satisfied if the patient parameter SpO2 equals or exceeds 85%. As shown, the actual value of SpO2 is 97 and the expression is satisfied (or true). Although acquisition of patient parameters is shown at 210, parameters may be acquired throughout the trial. Similarly, expressions may be evaluated throughout the trial.

At 230, the system automatically starts the SBT if start criteria is satisfied. In one example, SBT start criteria is satisfied when the success expressions are satisfied and none of the failure expressions are satisfied, at one instance in time, regardless of any duration requirements specified by the expressions. In another example, the system does not determine if the start criteria is satisfied. Instead, the system evaluates the success and failure expressions retroactively to determine if the duration requirements of the expressions were satisfied. The results may indicate that the automatic SBT was successful even though the SBT start was not made explicitly at the beginning of the trial.

At 240, the system determines if SBT success criteria is satisfied. The SBT success criteria may include success expressions and failure expressions. As described below, the expressions may comprise a protocol associated with a patient. The success and failure expressions may include functions and thresholds. The expressions may also include ranges. Ranges may also be defined by two expressions, each defining one of an upper and a lower limit of the range. Ranges may also be defined in a function comprising Boolean algebra, where the threshold is a logical outcome, e.g. yes/no. Expressions may also include time durations or compliance requirements. For example, an expression may be satisfied if a parameter exceeds a value, exceeds the value for a predetermined time during the trial, falls within a range for a predetermined time, etc.

At 242, a spontaneous breathing trial status indication is provided based on the evaluation of the expressions to provide constructive notice concerning the success or failure of the trial. In one example, constructive notice is given by indicating the satisfaction of each success expression and non-satisfaction of each failure expression, and also indicating a given time has lapsed, the given time representing a time period over which the satisfaction and non-satisfaction of the expressions is required to conduct a successful trial. Additional trial success criteria may also be provided apart from the satisfaction and non-satisfaction of the expressions.

In a variation of the present embodiment, a trial protocol status indicator is presented by the user interface. The time remaining in the trial may also be presented. Exemplary status indicators may comprise icons of different colors or shapes, flashing icons, text messages, audible indications, and any other means for providing constructive notice to the clinician concerning the success or failure of the trial.

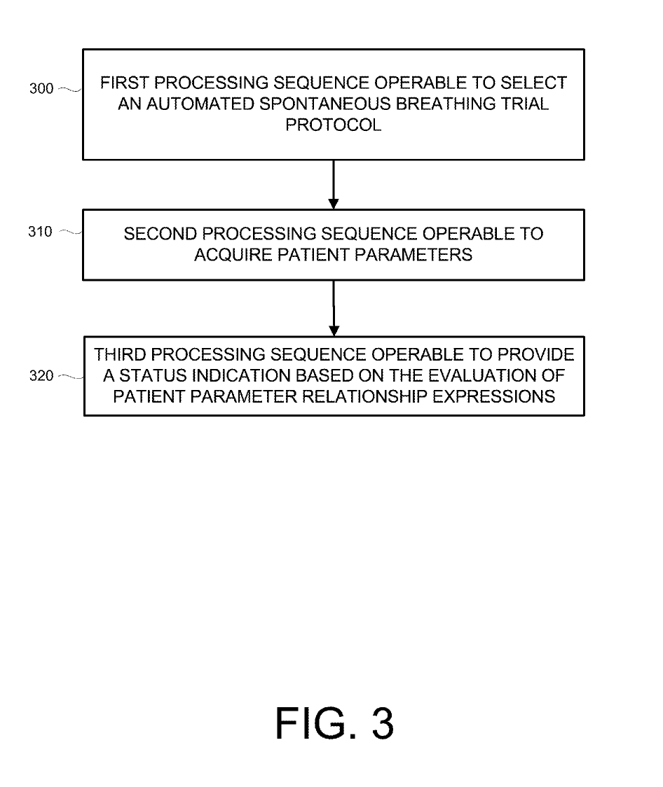

An embodiment according to the disclosure of a computer program product for performing an automated spontaneous breathing trial will now be described with reference to FIG. 3. The computer program product comprises a plurality of computer readable processing sequences embodied in a computer readable storage medium and operable to present views of a user interface with a display device and to receive user inputs associated with the views of the user interface, thereby enabling a user to manipulate information. Further, the computer program product may be operable to acquire patient parameters, evaluate expressions and update status indicators to enable the clinician to visually evaluate the progress of the spontaneous breathing trial. The expressions and protocols may be predefined. Also, the computer program product may be operable to add, modify and delete expressions and protocols.

A first processing sequence 300 is operable by a user to select a spontaneous breathing trial protocol including a plurality of patient parameter relationship expressions. Any number patient parameter relationship expressions and protocols may be predefined based on experience or medical literature. In one variation of the present embodiment, a single protocol is predefined and, therefore, a protocol does not need to be selected. In another variation of the present embodiment, no protocols are predefined and, therefore, a protocol is created rather than selected.

First processing sequence 300 may store the user selection in a configuration file such as a text file, an XML file or any other suitably formatted data structure. The user selection may be stored in a file corresponding to a specific patient. The user selection may also be stored in a database in which the selection is related or associated with the patient. In one variation, first processing sequence 300 presents a selection tool to enable the user to select a configuration file from memory. Once the user selects a configuration file, first processing sequence 300 may receive further user selections corresponding to additions or modifications of the patient parameter relationship expressions for the patient. First processing sequence 300 then stores the modified information in a database record or a patient configuration file, for example.

A second processing sequence 310 is operable to acquire the patient parameters. As described above, patient parameters may be acquired at periodic time intervals, based on the patient's physiology, or upon occurrence of some predetermined event.

A third processing sequence 320 is operable to provide a status indication based on the evaluation of the expressions to provide constructive notice concerning the success or failure of the trial. In one variation, third processing sequence 320 is also operable to determine a start criteria of the SBT trial, and the status indication may thus indicate automatic commencement of the SBT trial.

In a variation of the present embodiment, a trial protocol status indicator is presented by the user interface. The time remaining in the trial may also be presented. In a further variation of the present embodiment, expression status indicators are presented by the user interface to indicate the present status of the expressions.

In another embodiment according to the disclosure, a successful trial is indicated when the success expressions shown in Table A (below) are concurrently satisfied for at least a first time period. The trial may begin automatically upon the concurrent satisfaction of all the expressions. An exemplary time period is at least about an hour.

TABLE A

| PARAMETER | ACRONYM | OPERATOR | THRESHOLD |
|---|---|---|---|
| Time on Ventilator | | > | 24 hrs |
| # Neuromuscular blocking | | = | 0 |
| agents | | | |
| Positive end expiratory | PEEP | ≦ | 8 cm H₂0 |
| pressure | | | |
| Fraction of inspired oxygen | Fi02 | ≦ | 0.55 |
| Heart rate | HR | ≦ | 120 beats per |
| | | | minute |
| Oxygen saturation | Sp02 | ≧ | 90% |
| measured by pulse | | | |
| oximetry | | | |
| Temperature | | ≦ | 38.6° C. |
| Systolic blood pressure | SBP | ≧ | 90 mmHg |
| Minute ventilation | Ve | ≦ | 10 L |

The time-on-ventilator parameter may be satisfied if the patient is on the ventilator for 24 hours prior to the commencement of the SBT. In another example, where the SBT trial performance is assessed retroactively, the time-on-ventilator parameter may be satisfied if the patient has been on the ventilator for 24 hours at the time all the other success expressions are satisfied. Referring to Table A, the SBT may start, if the patient has been on the ventilator for 24 hours and no neuromuscular blocking agents have been administered to the patient, when the remaining parameters are satisfied. In one variation, if the parameters are satisfied with 90% compliance during one hour, the patient remains on the ventilator, and no neuromuscular blocking agents have been administered to the patient, then the SBT is successful.

In a further embodiment according with the disclosure, the success criteria may be further refined by inclusion of NIF and CORE success expressions, as shown below:

| Negative inspiratory force (maximum | NIF | ≦ | −20 cm H₂0 |
|---|---|---|---|
| inspiratory pressure) | | | |
| CORE index | CORE | > | 8 |

The CORE index is equal to: [C_{dyn}×P_{Imax}/P_{0.1}×P_{aO2}/P_{AO2})]/f, where
C_{dyn}=dynamic compliance (ml per cm H₂O)
P_{Imax}=maximum inspiratory pressure
P_{0.1}=airway occlusion pressure 0.1 seconds after start if inspiratory flow
P_{aO2}/P_{AO2}=arterial/alveolar partial pressure of oxygen ratio
f=respiratory rate (breaths per minute)

As the NIF success expression indicates, NIF improves as the value becomes more negative. A NIF of −30 is better than a NIF of −20 even though the value appears to be smaller (more negative). The symbol merely represents that the pressure being generated by the patient is negative, i.e. vacuum. Thus, a larger magnitude indicates that the patient is able to generate more pressure, and the negative symbol indicates that the pressure is vacuum.

In a further embodiment according with the disclosure, a successful trial is indicated when the success expressions shown in Table A are concurrently satisfied for at least the first time period and none of the failure expressions shown in Table B (below) are satisfied during the same period. If at least one of the failure expressions is satisfied, the time is reset. The trial may automatically begin again when the success expressions are satisfied if none of the failure expressions are satisfied.

TABLE B

| PARAMETER | OPERATOR | THRESHOLD |
|---|---|---|
| Change in systolic pressure | > | 30 mmHg |
| Change in diastolic pressure | > | 10 mmHg |
| Heart rate increase over baseline | > | 20 beats per |
| for at least 5 continuous minutes | | minute |

In a further embodiment according with the disclosure, the failure criteria may be further refined by inclusion of the PCO2 failure expression, as shown below:

| | | |
|---|---|---|
| Increase in partial pressure of carbon | > | 5 mmHg |
| dioxide (PCO2) | | |

In a yet further embodiment according to the disclosure, a protocol includes the expressions shown in Table C (below). Prior to the trial, the ventilator settings may be set as follows:
Mode=spontaneous or adaptive
PEEP≦7
FiO2<55%

It should be understood that ventilator settings are set based on the particularities of the ventilator, the patient and the patient conditions. In another example, the FiO2 parameter may be set to 40%. In a further example, the FiO2 parameter may be set to 60%.

Success of an expression may be defined as a predetermined compliance during the trial period. Compliance is the ratio, expressed as a percentage, of the time an expression is satisfied over the trial duration. For example, 90% compliance, in a one hour trial, is achieved when an expression is satisfied for at least 54 minutes. The trial may be reset if a success expression cannot satisfy the compliance requirement. Referring to Table C below, selected expressions include a compliance requirement. For example, Heart rate—95% compliance—indicates that the heart rate must be 120 beats per minute at least 95% of the trial period. Similarly, Respiratory rate—95% compliance—indicates that the respiratory rate must be <28 breaths per minute at least 95% of the trial period. Some expressions do not include an explicit compliance requirement. For example, the Time on Ventilator parameter must exceed 24 hours during the trial. Thus, the patient must be on the ventilator for at least 24 hours during the entire trial, meaning that if the patient is taken off the ventilator during the trial or the preceding 24 hours, the expression is not satisfied. Implicitly then, the compliance requirement is 100% unless otherwise stated.

TABLE C

| | | |
|---|---|---|
| PARAMETER | OPERATOR | THRESHOLD |

SUCCESS EQUATIONS

| | | |
|---|---|---|
| Time on Ventilator | > | 24 hours |
| # Neuromuscular blocking agents | = | 0 |
| Ppeak—PEEP | ≦ | 8 cm H₂0 |
| Heart rate - 95% compliance | ≦ | 120 beats per |
| | | minute |
| Respiratory rate - 95% compliance | < | 28 breaths per |
| | | minute |
| SpO2—95% compliance | ≧ | 90% |
| Temperature | ≦ | 38.6° C. |
| Systolic blood pressure | ≧ | 90 mmHg |
| Minute ventilation - 90% compliance | ≦ | 10 L |
| RSBI (rapid shallow breathing index = | < | 105 |
| f/VT, where VT is the tidal volume) - | | |
| 90% compliance | | |

FAILURE EQUATIONS

| | | |
|---|---|---|
| Change in systolic pressure | > | 30 mmHg |
| Change in diastolic pressure | > | 10 mmHg |
| Heart rate increase over baseline for at | > | 20 beats per |
| least 5 continuous minutes | | minute |

In another embodiment, the success criteria may be further refined by inclusion of NIF and CORE success expressions, as shown above. In a further embodiment, the failure criteria may be further refined by inclusion of the PCO2 failure expression shown above.

Referring again to FIG. 1, system 100 comprises a computing device 104 including one or more processing device and software programs. As used herein, a software program, algorithm, or processing sequence, is a self consistent sequence of instructions that can be followed to perform a particular task. Software programs may use data structures for both inputting information and performing the particular task. Data structures greatly facilitate data management. Data structures are not the information content of a memory, rather they represent specific electronic structural elements which impart a physical organization on the information stored in memory. More than mere abstraction, the data structures are specific electrical or magnetic structural elements in memory which simultaneously represent complex data accurately and provide increased efficiency in computer operation.

A processing or computing system or device may be a specifically constructed apparatus or may comprise general purpose computers selectively activated or reconfigured by software programs stored therein. The computing device, whether specifically constructed or general purpose, has at least one processing device, for executing machine instructions, which may be grouped in processing sequences, and access to computer readable storage media, or memory, for storing instructions and other information. Many combinations of processing circuitry and information storing equipment are known by those of ordinary skill in these arts. A processing device may be a microprocessor, a digital signal processor (DSP), a central processing unit (CPU), or other circuit or equivalent capable of interpreting instructions or performing logical actions on information. Memory includes both volatile and non-volatile memory, including temporary and cache, in electronic, magnetic, optical, printed, or other format used to store information. Exemplary processing systems include workstations, personal computers, portable computers, portable wireless devices, mobile devices, and any device including a processor, memory and software. Processing systems encompass one or more computing devices and include computer networks and distributed computing devices.

As used herein, a computer network, or network, is a system of computing systems or computing devices interconnected in such a manner that messages may be transmitted between them. Typically one or more computers operate as a “server”, a computer with access to large storage devices such as hard disk drives and communication hardware to operate peripheral devices such as printers, routers, or modems. Other computers, termed “clients”, provide a user interface so that users of computer networks can access the network resources, such as shared data files, common peripheral devices, and inter workstation communication. User interfaces comprise software working together with user devices to communicate user commands to the processing system. Exemplary user devices include touch-screens, keypads, mice, voice-recognition logic, imaging systems configured to recognize gestures, and any known or future developed hardware suitable to receive user commands.

Embodiments of the disclosure may be implemented in “object oriented” software, and particularly with an “object oriented” operating system. The “object oriented” software is organized into “objects”, each comprising a block of computer instructions describing various procedures to be performed in response to “messages” sent to the object or “events” which occur with the object. Such operations include, for example, the manipulation of variables, the activation of an object by an external event, and the transmission of one or more messages to other objects. Messages are sent and received between objects having certain functions and knowledge to carry out processes. Messages are generated in response to user instructions, for example, by a user activating an icon with a mouse pointer or touch-screen to generate an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access.

Referring now to FIG. 4, a block diagram of another embodiment of an automated spontaneous breathing trial system according to the disclosure is presented. In the present embodiment, the system is denoted by numeral 400. As in system 100, system 400 may receive patient parameters from a plurality of data sources including a medical records database, patient monitoring devices, and user interfaces. As shown, system 400 comprises a data collection subsystem 410. Data collection subsystem 410 includes a data collection program 402, a computing device 404 and a patient parameter database 406. Data collection program 402 causes computing device 404 to acquire patient parameters from the data sources and to store the patient parameters in patient parameter database 406. Data collection subsystem 410 may also store monitoring device setting information. Patient parameters may be normalized prior to being stored in patient parameter database 406.

System 400 also comprises an expression management subsystem 420. Expression management subsystem 420 includes an expression management program 422, a computing device 424 and an expression database 426. Expression management program 422 may cause computing device 424 to acquire patient parameters from patient parameter database 406 and to store the patient parameters in a computer readable storage medium.

System 400 also comprises an SBT evaluation subsystem 440. As shown, SBT evaluation subsystem 440 comprises user interface 116, display device 130 and a computing device 444. SBT evaluation subsystem 440 may be operable to communicate with expression management program 422 information relating to the patient parameter relationship expressions and patient parameters. SBT evaluation subsystem 440 may then determine the status of the patient parameter relationship expressions and the spontaneous breathing trial. User interface 116 may be operable to cause computing device 444 to receive user instructions, such as protocol and expression selections, and to configure and modify expressions and protocols as described above and further below.

In a further embodiment of an automated spontaneous breathing trial system according with the disclosure, data collection subsystem 400, expression management subsystem 420 and SBT evaluation subsystem 440 are configured in a client/server architecture. In one example, data collection subsystem 400 is integrated in a hospital environment and may acquire patient information as determined by the data acquisition parameters of the data sources. Expression management subsystem 420 pulls and stores patient parameters from data collection subsystem 400. Thus, by mapping protocol parameters, expression management subsystem 420 may be linked to a data collection subsystem 400 which forms part of a hospital's data infrastructure. Furthermore, SBT evaluation subsystem 440 may be communicatively linked to expression management subsystem 420 to provide a visual aid to a clinician near the patient. Expression management subsystem 420 may function as a server to support a plurality of SBT evaluation subsystem 440 clients.

In one variation of the present embodiment, expression management program 422 may also determine the status of the expressions and store the status of the expressions in expression database 426. In the present variation, SBT evaluation subsystem 440 may receive the status information and provide trial status indications accordingly.

In a further embodiment of an automated spontaneous breathing trial system, data collection subsystem 410 includes expression database 426 and expression management subsystem 420 is not required. In the present embodiment, SBT evaluation subsystem 440 is communicatively coupled with data collection subsystem 410.

An automated spontaneous breathing trial system according with the disclosure may present a plurality of views with the user interface. Each view may be presented on a separate tab. A view may include a drop-down list of patients that may be selected to enable a clinician to switch the presentation of information for different patients, a drop-down list of protocols to enable a clinician to quickly switch from one protocol to another, data entry fields to enter patient parameters, and one or more indicators to provide constructive notice of the success or failure of the spontaneous breathing trial.

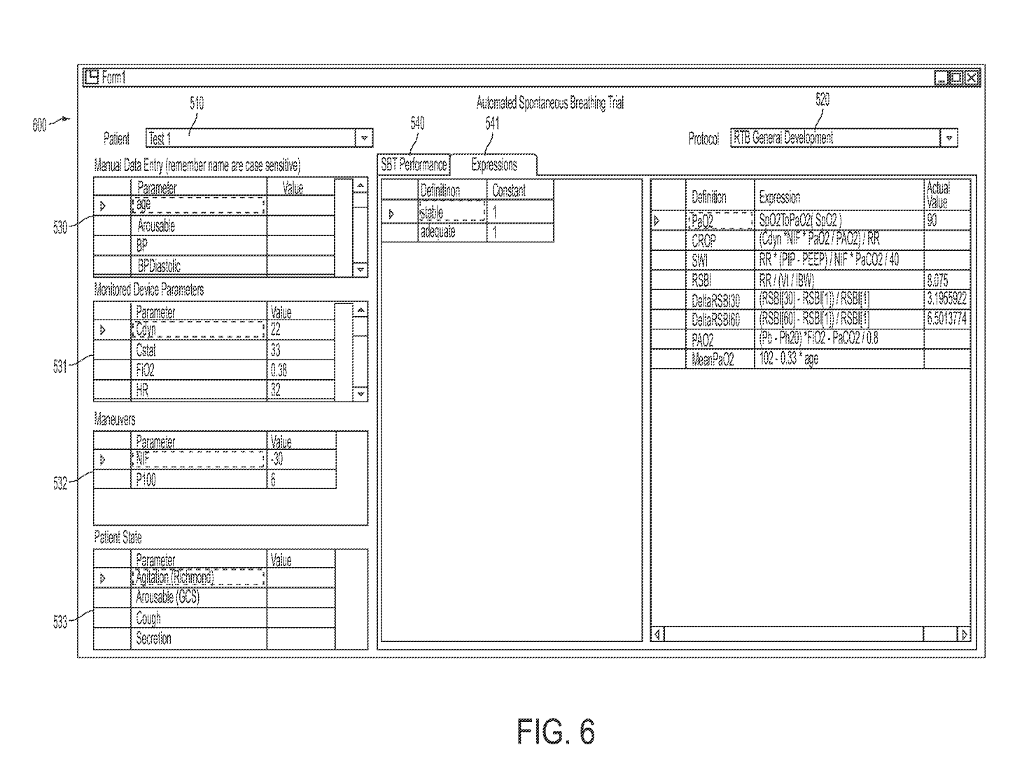

Exemplary user interface views are presented on FIGS. 5 and 6. FIG. 5 presents a user interface view 500 comprising a patient selection box 510, a protocol selection box 520, a plurality of patient parameter panels 530-533, and tabs 540 and 541. Tab 540, corresponding to the spontaneous breathing trial, is selected. Patient selection box 510 and protocol selection box 520 enable users to select patients and protocols. The selections may be stored in patient specific configuration files on expression database 426 or on any other suitable storage location. Patient parameter panels 530-533 display patient parameters corresponding to, respectively, manual data entry, monitored devices, maneuvers, and patient states. A user may enter manually a patient parameter for parameters shown in panels 530, 532 and 533.

Referring to FIG. 5, tab 540 includes a success expressions table 550 and may include a failure expressions table (not shown). Table 550 includes a plurality of patient parameter relationship expressions and also a column labeled “pass” showing the status indicators corresponding to the present status of the expressions. The plurality of expressions represent a protocol, e.g. protocol titled “2001 Evidence Based Guidelines”. Each expression is presented as a line item, detailing the expression, calculated result of the expression, the condition specified, and the threshold (target) value. As shown, status indicators include text (true/false) and a color indicative of success (evidenced by colored “True” status indicators in table 550). Satisfaction of an expression may be highlighted green, failure may be highlighted red, and unable to compute may be left plain.

In a variation of the present embodiment, a protocol status indicator is displayed to provide constructive notice with respect to the success or failure of the protocol. In one exemplary embodiment disclosed on FIG. 5, three status indicators are provided. One status indicator is the time remaining in the SBT. In the present example, the time remaining is shown as 2:00:00 next to the label “SBT Time Remaining:” to indicate that two hours remain in the trial. A checkbox next to the label “SBT Tolerated” is provided which would automatically show a checkmark if the trial had been successful. A checkbox next to the label “SBT Failed” is provided which would automatically show a checkmark if the trial had failed. Since two hours remain in the trial, the trial is not yet successful even though it has also not failed. In another example, tab 540 displays the SBT Tolerated checkbox and the SBT Failed checkbox, which a clinician may check to document the success or failure of the SBT. The clinician may determine that the SBT was successful if there is no time remaining in the SBT and the system did not indicate an SBT failure.

In a still further variation of the present embodiment, a selection tool is presented (not shown) to enable a clinician to select a patient parameter or expression. The system then presents a graphical representation comprising historical values of the patient parameter or the expression.

Referring now to FIG. 6, a user interface view 600 is presented with a tab labeled “Expressions” selected. The expressions tab comprises tables of constants and variables. The constants table shows the values assigned to named constants. For example, constants named stable and adequate are assigned the value “1”. In the variables table, a plurality of variables and corresponding expressions are shown. The first variable in the table is named PaO2. The value of PaO2 is defined by the corresponding expression (function) shown next to the variable name, the PaO2 variable having an actual value equal to 90. The second variable in the table is named CROP. CROP is a composite expression in that it includes variable PaO2. The constants and variables may transform parameters from data sources into forms more suitable for use in the patient parameter relationship expressions. The expressions may be defined and transformed to match clinical thought and the styles used in medical literature and references. For example, the variables may scale and normalize parameters. Also, the variables may consolidate parameters from different sources to simplify configuration of the patient parameter relationship expressions and protocols.

While this invention has been described as having an exemplary design, the present invention may be further modified within the spirit and scope of this disclosure. This application is intended to cover any variations, uses, or adaptations of the invention using its general principles. Further, this application is intended to cover such departures from the present disclosure as come within known or customary practice in the art to which this invention pertains.

What is claimed is:

1. A method implemented with a computing system to automatically perform a spontaneous breathing trial, the method comprising:
acquiring a plurality of patient parameters of a plurality of patient parameter relationship expressions stored in a computer readable storage medium;
evaluating the plurality of patient parameter relationship expressions;
determining if a success criteria is satisfied, the success criteria based at least in part on satisfaction of at least some of the plurality of patient parameter relationship expressions for a given time; and
determining that the spontaneous breathing trial is successful if the success criteria is satisfied.

2. A method as in claim 1, further comprising:
automatically starting the spontaneous breathing trial when a start criteria is satisfied; and
if the spontaneous breathing trial is successful, providing constructive notice of the success with a user interface.

3. A method as in claim 1, wherein the patient parameter relationship expressions include success expressions and failure expressions, and wherein the success criteria is based at least in part on satisfaction of the success expressions and non-satisfaction of all the failure expressions during the given time.

4. A method as in claim 3, wherein the success criteria comprises satisfaction of all the success expressions with at least about 90% compliance and non-satisfaction of all the failure expressions.

5. A method as in claim 3, wherein the given time is about one hour.

6. A method as in claim 3, wherein the success expressions comprise:

| | | |
|---|---|---|
| Time on Ventilator | > | 24 hours |
| # Neuromuscular blocking agents | = | 0 |
| Ppeak - PEEP | ≦ | 8 cm H₂0 |
| Heart rate - 95% compliance | ≦ | 120 beats per |
| | | minute |
| Respiratory rate - 95% compliance | < | 28 breaths per |
| | | minute |
| SpO2 - 95% compliance | ≧ | 90% |
| Temperature | ≦ | 38.6° C. |
| Systolic blood pressure | ≧ | 90 mmHg |
| Minute ventilation - 90% compliance | ≦ | 10 L |
| RSBI (rapid shallow breathing index = f/VT, | < | 105 |
| where VT is the tidal volume) - 90% | | |
| compliance | | |

and the failure expressions comprise:

| | | |
|---|---|---|
| Change in systolic pressure | > | 30 mmHg |
| Change in diastolic pressure | > | 10 mmHg |
| Heart rate increase over baseline for at least | > | 20 beats per |
| 5 continuous minutes | | minute |

7. A method as in claim 1, further comprising:
selecting a protocol from a plurality of protocols with a user interface, the protocol including the plurality of patient parameter relationship expressions.

8. A computing system to automatically perform a spontaneous breathing trial, the system comprising:
a computer readable storage medium having stored therein a plurality of patient parameter relationship expressions based on patient parameters;
a computing device, coupled to the computer readable storage medium, that is configured to (a) acquire the patient parameters; (b) evaluate the plurality of patient parameter relationship expressions; (c) determine if a success criteria is satisfied, the success criteria based at least in part on satisfaction of at least some of the plurality of patient parameter relationship expressions for a given time; and (d) determine that the spontaneous breathing trial is successful if the success criteria is satisfied.

9. A computing system as in claim 8, the computing device further configured to automatically start the spontaneous breathing trial when a start criteria is satisfied, and the computing system further comprising:
a user interface stored in the computer readable storage medium and configured to, if the spontaneous breathing trial is successful, providing constructive notice of the success.

10. A computing system as in claim 8, wherein the patient parameter relationship expressions include success expressions and failure expressions, and wherein the success criteria is based at least in part on satisfaction of the success expressions and non-satisfaction of all the failure expressions during the given time.

11. A computing system as in claim 10, wherein the success criteria comprises satisfaction of all the success expressions with at least about 90% compliance and non-satisfaction of all the failure expressions.

12. A computing system as in claim 10, wherein the given time is about one hour.

13. A computing system as in claim 10, wherein the success expressions comprise:

| | | |
|---|---|---|
| Time on Ventilator | > | 24 hours |
| # Neuromuscular blocking agents | = | 0 |
| Ppeak - PEEP | ≦ | 8 cm H₂0 |
| Heart rate - 95% compliance | ≦ | 120 beats per |
| | | minute |
| Respiratory rate - 95% compliance | < | 28 breaths per |
| | | minute |
| SpO2 - 95% compliance | ≧ | 90% |
| Temperature | ≦ | 38.6° C. |
| Systolic blood pressure | ≧ | 90 mmHg |
| Minute ventilation - 90% compliance | ≦ | 10 L |
| RSBI (rapid shallow breathing index = f/VT, | < | 105 |
| where VT is the tidal volume) - 90% | | |
| compliance | | |

and the failure expressions comprise:

| | | |
|---|---|---|
| Change in systolic pressure | > | 30 mmHg |
| Change in diastolic pressure | > | 10 mmHg |
| Heart rate increase over baseline for at least | > | 20 beats per |
| 5 continuous minutes | | minute |

14. A computing system as in claim 8, further comprising:
selecting a protocol from a plurality of protocols stored in the computer readable storage medium with a user interface, the protocol including the plurality of patient parameter relationship expressions.

15. A computer program product comprising a computer readable storage medium having computer readable processing sequences embodied therein for causing a computing system to automatically perform a spontaneous breathing trial, the computer program product comprising:
a first processing sequence operable to acquire patient parameters of a plurality of patient parameter relationship expressions;
a second processing sequence operable to evaluate the plurality of patient parameter relationship expressions;
a third processing sequence operable to determine if a success criteria was satisfied, the success criteria based at least in part on satisfaction of at least some of the plurality of patient parameter relationship expressions for a given time; and
a fourth processing sequence operable to determine that the spontaneous breathing trial is successful if the success criteria is satisfied.

16. A computer program product as in claim 15, further comprising a processing sequence operable to automatically start the spontaneous breathing trial when a start criteria is satisfied and, if the spontaneous breathing trial is successful, to provide constructive notice of the success.

17. A computer program product as in claim 15, wherein the patient parameter relationship expressions include success expressions and failure expressions, and wherein the success criteria is based at least in part on satisfaction of the success expressions and non-satisfaction of all the failure expressions during the given time.

18. A computer program product as in claim 17, wherein the success criteria comprises satisfaction of all the success expressions with at least about 90% compliance and non-satisfaction of all the failure expressions.

19. A computer program product as in claim 17, wherein the given time is about one hour.

20. A computer program product as in claim 17, wherein the success expressions comprise:

| | | |
|---|---|---|
| Time on Ventilator | > | 24 hours |
| # Neuromuscular blocking agents | = | 0 |
| Ppeak - PEEP | ≦ | 8 cm H₂0 |
| Heart rate - 95% compliance | ≦ | 120 beats per |
| | | minute |
| Respiratory rate - 95% compliance | < | 28 breaths per |
| | | minute |
| SpO2 - 95% compliance | ≧ | 90% |
| Temperature | ≦ | 38.6° C. |
| Systolic blood pressure | ≧ | 90 mmHg |
| Minute ventilation - 90% compliance | ≦ | 10 L |
| RSBI (rapid shallow breathing index = f/VT, | < | 105 |
| where VT is the tidal volume) - 90% | | |
| compliance | | |

and the failure expressions comprise:

| | | |
|---|---|---|
| Change in systolic pressure | > | 30 mmHg |
| Change in diastolic pressure | > | 10 mmHg |
| Heart rate increase over baseline for at least | > | 20 beats per |
| 5 continuous minutes | | minute |
